(19) United States
(12) Patent Application Publication
(10) Pub. No.: US 2017/0347903 A1
(43) Pub. Date: Dec. 7, 2017

(54) BIOLOGICAL INFORMATION DISPLAY SYSTEM, PORTABLE TERMINAL APPARATUS, WEARABLE APPARATUS, BIOLOGICAL INFORMATION DISPLAY METHOD, AND BIOLOGICAL INFORMATION DISPLAY PROGRAM

(71) Applicant: SEIKO EPSON CORPORATION, Tokyo (JP)

(72) Inventor: Shintaro NAGASAKI, Hara-mura (JP)

(73) Assignee: SEIKO EPSON CORPORATION, Tokyo (JP)

(21) Appl. No.: 15/595,201

(22) Filed: May 15, 2017

(30) Foreign Application Priority Data
Jun. 1, 2016 (JP) 2016-109808

(51) Int. Cl. A61B 5/024; A61B 5/0205; A61B 5/11; A61B 5/00

(52) U.S. Cl. CPC A61B 5/02438; A61B 5/02405; A61B 5/743; A61B 5/7225; A61B 5/681; A61B 5/0205; A61B 5/1118; A61B 5/02416; A61B 5/7285; A61B 5/4806

(57) ABSTRACT

A biological information display system includes a display unit, a pulse information detecting unit that obtains biological information of a user, a control unit that calculates a pulse rate of the user based on the biological information, and determines analysis information based on the calculated pulse rate, and causes the analysis information, and time information responding to the analysis information to be displayed in the display unit by being linked to each other, and an operation accepting unit that accepts input information including at least one of subjective information relating to the user and event information relating to the user by associating the input information with the time information, in which in a case where the operation accepting unit accepts the input information, the control unit causes the input information to be displayed in the display unit by being linked to the analysis information and the time information.

### CROSS-REFERENCES TO RELATED APPLICATIONS

This application claims priority to Japanese Patent Application No. 2016-109808, filed Jun. 1, 2016, the entirety of which is herein incorporated by reference.

### BACKGROUND

### 1. Technical Field

The present invention relates to a biological information display system, a portable terminal apparatus, a wearable apparatus, a biological information display method, and a biological information display program.

### 2. Related Art

Heretofore, an apparatus that obtains pulse rate information of a user by a sensor, and determines a health state of the user based on the obtained pulse rate information is provided. For example, an apparatus disclosed in JP-A-2015-131049 detects a pulse rate of the user, and displays a minimum value of the pulse rate by calculating the minimum value thereof at the time of sleeping, based on the detected pulse rate. Thereby, the user can visually recognize the minimum value of the pulse rate.

In general, it is said that the minimum value of the pulse rate has a correlation with a body condition, Therefore, the user can realize a change of the body condition by realizing transition of the minimum value of the pulse rate by lapse of time.

In the apparatus of the related art, the user can realize the transition of the minimum value of the pulse rate, but the transition thereof is not linked to a behavior or an emotional change of the user. Therefore, it is necessary for the user to estimate a change factor in the body condition or the like responding to the change of the minimum value of the pulse rate based on memory of the user, and the accurate estimation. is difficult since the memory of the past is vague.

### SUMMARY

An advantage of some aspects of the invention is to easily estimate a factor that seems to influence a body condition in transition of a minimum value of a pulse rate.

The invention can be implemented as the following forms or application examples.

### APPLICATION EXAMPLE 1

A biological information display system according to this application example includes a display unit, biological information obtaining unit that obtains biological information of a user, a control unit that determines analysis information relating to a state of the user based on the biological information, and causes the analysis information, and time information responding to the analysis information to be displayed in the display unit by being linked to each other, and an accepting unit that accepts input information including at least one of subjective information relating to the user and event information relating the user by associating the input information with the time information, in which in a case where the accepting unit accepts the input information, the control unit causes the input information to be displayed in the display unit by being linked to the analysis information and the time information.

According to this configuration, in a case where the input information including at least one of the subjective information relating to the user and the event information relating the user is input by being linked to the time information, the input information is displayed in the display unit by being linked to the time information, and the analysis information of the user which is linked to the time information. Therefore, the user can accurately estimate a factor for the analysis information, based on the input information.

### APPLICATION EXAMPLE 2

In the biological information display system according to the application example, it is preferable that the control unit causes a plurality of pieces of the analysis information to be displayed in a time series depending on respective pieces of the time information.

According to this configuration, since the analysis information is displayed in the time series depending on the time information, the user can visually recognize transition of the analysis information in the time series.

### APPLICATION EXAMPLE 3

In the biological information display system according to the application example, it is preferable that the input information is information relating to at least one of drinking, a body condition, exercise, a mental state, sleep time and sleep depth.

According to this configuration, the user can estimate the factor of the analysis information which is linked to the time information by linking the factor thereof to at least one of the drinking, the body condition, the exercise, the mental state, the sleep time and the sleep depth.

### APPLICATION EXAMPLE 4

In the biological information display system according to the application example, it is preferable that the biological information is at least one of pulse wave information, electrocardiac information and a pulse rate.

According to this configuration, it is possible to calculate the pulse rate of the user from at least one of the pulse wave information and the electrocardiac information.

### APPLICATION EXAMPLE 5

In the biological information display system according to the application example, the analysis information may be at least any one of a minimum pulse rate, a resting pulse rate, a maximum pulse rate, a stress degree and a fatigue degree.

### APPLICATION EXAMPLE 6

In the biological information display system according to the application example, it is preferable that the control unit assigns the analysis information to one axis of a graph, and the time information to the other axis of the graph, and generates a transition image indicating transition of the analysis information by lapse of time, and causes the transition image to be displayed in the display unit.

According to this configuration, it is possible to visually recognize the transition of the analysis information with ease by indicating the transition of the analysis information by the transition image where the analysis information is assigned to one axis of the graph, and the time information is assigned to the other axis.

### APPLICATION EXAMPLE 7

In the biological information display system according to the application example, it is preferable that the control unit indicates the analysis information depending on the time information by a plot in the transition image, and causes an icon depending on the input information to be displayed by being superimposed on the plot, in a case where the input information is linked to the time information.

According to this configuration, in a case where the analysis information is indicated by the plot in the transition image, and the input information is linked to the time information, since the icon depending on the input information is displayed by being superimposed on the plot, it is possible to visually estimate the factor for the transition of the analysis information, by visually recognizing the icon responding to the analysis information.

### APPLICATION EXAMPLE 8

In the biological information display system according to the application example, it is preferable that the analysis information is a minimum pulse rate, and the control unit determines a basal pulse rate of the user based on transition of the minimum pulse rate, and indicates a base line indicating the basal pulse rate on the transition image.

According to this configuration, it is possible to visually compare the basal pulse rate with the analysis information, by indicating the basal pulse rate of the user as a base line on the transition image.

### APPLICATION EXAMPLE 9

In the biological information display system according to the application example, it is preferable that the analysis information is a minimum pulse rate, and the control unit obtains the minimum pulse rate in a case where a body condition or a sleep state of the user is fine, and indicates a base line indicating the minimum pulse rate on the transition image.

According to this configuration, it is possible to visually compare the analysis information in a case where the body condition or the sleep state is fine with the current analysis information, by indicating the analysis information as a base line on the transition image in a case where the body condition or the sleep state of the user is fine.

### APPLICATION EXAMPLE 10

In the biological information display system according to the application example, it is preferable that the control unit determines a fatigue degree of the user, based on a divergence degree between the analysis information and the base line.

According to this configuration, it is possible to determine the fatigue degree of the user by detecting the divergence degree between the obtained analysis information and the base line, and to present the fatigue degree to the user.

### APPLICATION EXAMPLE 11

In the biological information display system according to the application example, it is preferable that the control unit includes a notification unit that notifies the user of a message, based on the determined fatigue degree and the input information.

According to this configuration, since the notification unit notifies of the message, based on the fatigue degree determined by the control unit and the input information, it is possible to notify the user of the fatigue degree.

### APPLICATION EXAMPLE 12

In the biological information display system according to the application example, it is preferable that the control unit evaluates a similarity degree between a first change pattern indicating a time series change of the analysis information and a second change pattern of the analysis information including most recent information, and predicts a change relating to a fatigue degree of the user, based on the similarity degree.

According to this configuration, since the change relating to the fatigue degree of the user is predicted, based on the similarity degree between the second change pattern indicating the time series change of the analysis information and the first change pattern stored in a storage unit, it is possible to prompt the user to appropriately respond.

### APPLICATION EXAMPLE 13

A portable terminal apparatus according to this application example includes a display unit, a receiving unit that receives biological information of a user, a control unit that calculates a pulse rate of the user based on the biological information, and determines analysis information based on the calculated pulse rate, and causes the analysis information, and time information responding to the analysis information to be displayed in the display unit by being linked to each other, and an accepting unit that accepts input information including at least one of subjective information relating to the user and event information relating to the user by associating the input information with the time information, in which in a case where the accepting unit accepts the input information, the control unit causes the input information to be displayed in the display unit by being linked to the analysis information and the time information.

According to this configuration, in a case where the input information including at least one of the subjective information relating to the user and the event information relating to the user is input by being linked to the time information, the input information is displayed in the display unit by being linked to the time information, and the analysis information of the user which is linked to the time information. Therefore, the user can accurately estimate the factor for the analysis information, based on the input information.

### APPLICATION EXAMPLE 14

A wearable apparatus according to this application example includes a display unit, a biological information obtaining unit that obtains biological information of a user, a control unit that determines analysis information relating to a state of the user based on the biological information, and causes the analysis information, and time information responding to the analysis information to be displayed in the display unit by being linked to each other, and an accepting unit that accepts input information including at least one of subjective information relating to the user and event information relating to the user by associating the input information with the time information, in which in a case where the accepting unit accepts the input information, the control unit causes the input information to be displayed in the display unit by being linked to the analysis information and the time information.

According to this configuration, in a case where the input information including at least one of the subjective information relating to the user and the event information relating to the user is input by being linked to the time information, the input information is displayed in the display unit by being linked to the time information, and the analysis information of the user which is linked to the time information. Therefore, the user can accurately estimate the factor for the analysis information, based on the input information.

### APPLICATION EXAMPLE 15

A biological information display method according to this application example includes obtaining biological information of a user, determining analysis information relating to a state of the user based on the biological information, and displaying the analysis information, and time information responding to the analysis information by linking the analysis information to the time information, and displaying input information by linking the input information to the analysis information and the time information, in a case of accepting the input information including at least one of subjective information relating to the user and event information relating to the user by linking the input information to the time information.

According to this method, in a case where the input information including at least one of the subjective information relating to the user and the event information relating to the user is input by being linked to the time information, the input information is displayed by being linked to the time information, and the analysis information of the user which is linked to the time information. Therefore, the user can accurately estimate the factor for the analysis information, based on the input information.

### APPLICATION EXAMPLE 16

A biological information display program according to this application example causes a computer to execute obtaining biological information of a user, determining analysis information relating to a state of the user based on the biological information, and displaying the analysis information, and time information responding to the analysis information by linking the analysis information to the time information, and displaying input information by linking the input information to the analysis information and the time information, in a case of accepting the input information including at least one of subjective information relating to the user and event information relating to the user by associating the input information with the time information.

According to this configuration, in a case where the input information ding at least one of the subjective information relating to the user and the event information relating to the user is input by being linked to the time information, the input information is displayed by being linked to the time information, and the analysis information of the user which is linked to the time information. Therefore, the user can accurately estimate a factor for the analysis information, based on the input information.

### BRIEF DESCRIPTION OF THE DRAWINGS

The invention will be described with reference to the accompanying drawings, wherein like numbers reference like elements.

FIG. 1 is a diagram illustrating an example of a biological information display system according to an embodiment

FIG. 2 is a diagram illustrating a configuration example of functional units of the biological information display system.

FIG. 3 is a diagram illustrating a transition image of a minimum pulse rate.

FIG. 4 is a flowchart of processing which is executed in a case where the minimum pulse rate of the transition image is tapped.

FIG. 5 is a diagram illustrating the transition image of the minimum pulse rate.

FIG. 6 is a diagram illustrating the transition image of the minimum pulse rate.

FIG. 7 is a diagram illustrating the transition image of the minimum pulse rate.

FIG. 8 is a diagram illustrating the transition image of the minimum pulse rate.

### DESCRIPTION OF EXEMPLARY EMBODIMENTS

Hereinafter, embodiments of the invention will be described with reference to the drawings.

### Embodiments

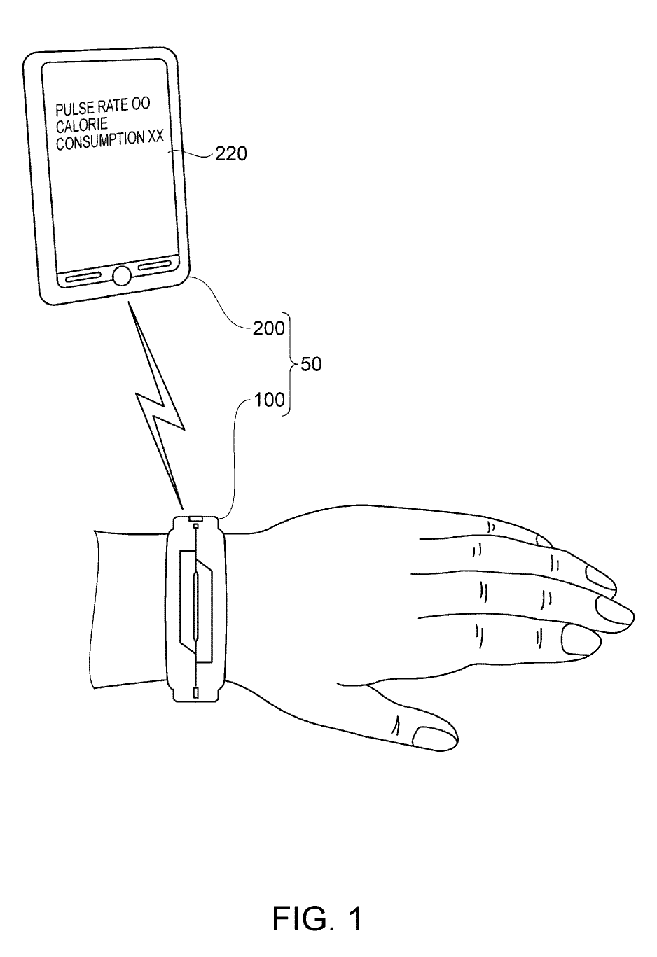

FIG. 1 is a diagram illustrating an example of a biological information display system 50 according to an embodiment. In FIG. 1, a band type wearable apparatus 100 is connected to a portable terminal apparatus 200 such as a smartphone by short-range wireless communication or the like, and information that is calculated based on the information from a sensor mounted in the wearable apparatus 100 is displayed in a display unit 220 of the portable terminal apparatus 200.

An electronic device according to the embodiment is not limited to the wearable apparatus 100 or the portable terminal apparatus 200, and a multifunction portable terminal such as a tablet or various devices such as a personal computer may be used.

The wearable apparatus 100 is not limited to the band type such as a wristwatch, and may be assumed to be various types of a pendant type, a belt type, and a smart glass type.

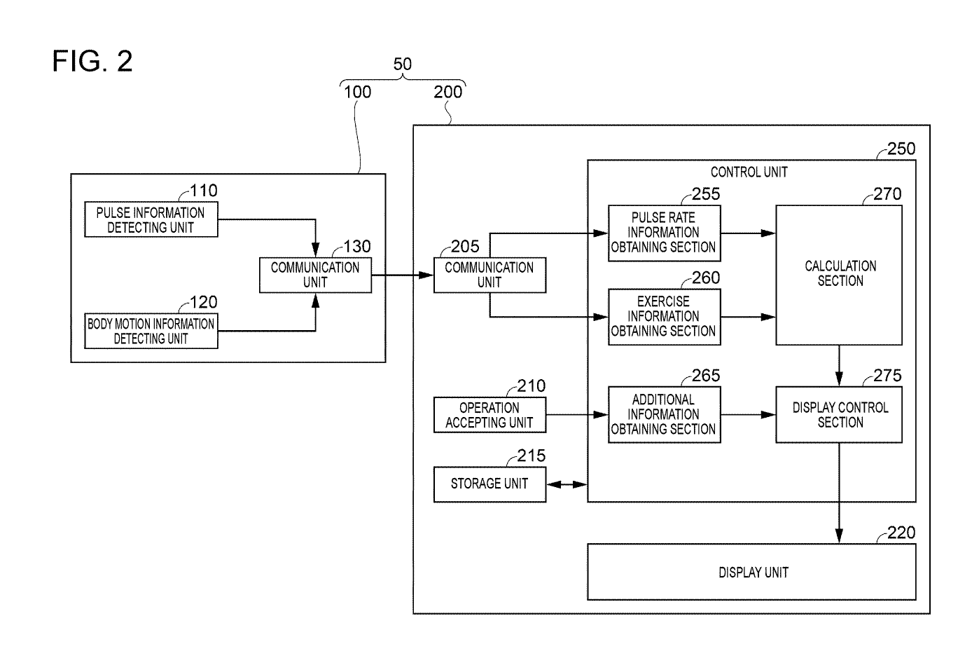

FIG. 2 illustrates a configuration example of functional units of the biological information display system 50 according to the embodiment.

First, the functional units of the wearable apparatus 100 will be described.

The wearable apparatus 100 includes a pulse information detecting unit 110, a body motion information detecting unit 120, and a communication unit 130.

The pulse information detecting unit 110 obtains pulse information including a pulse rate per unit time, based on an output of a pulse wave sensor (not illustrated) that detects a pulse wave being one sort of biological information. Moreover, the pulse information detecting unit 110 is equivalent to a biological information obtaining unit. For example, the pulse wave sensor includes a light-emitting element such as a light emitting diode (LED), and a light-receiving element such as a photodiode, The light irradiated by the light-emitting element is reflected by blood vessels of a body portion, and the reflected light is collected, and is received by the light-receiving element. At that time, the pulse wave sensor detects the pulse wave from change information of a light-receiving amount by using a phenomenon in which the reflectances of the light are different from each other at the time of dilation and constriction of the blood vessels.

For example, a method disclosed in JP-A-2012-232010 may be adopted in the method for calculating e pulse rate based on the reflected light from the blood vessels of the body portion.

In the embodiment, the pulse rate is detected as biological information, but it is not limited thereto. Electrocardiac information may be detected, and a heart rate may be detected based on the detected electrocardiac information. Moreover, the pulse wave information, and the electrocardiac information may be also detected. In the following description, the pulse rate may be replaced with the heart rate.

The body motion information detecting unit 120 obtains body motion information such as the number of steps based on an output of a sensor (not illustrated) that detects a body motion. The sensor is assumed to be an acceleration sensor, and a gyro sensor.

The acceleration sensor is an acceleration sensor that is attached into one axial direction or two or more axial directions (for example, three axial directions of X-axis, Y-axis, and Z-axis), and measures an acceleration change of each axis per unit time.

For example, a method disclosed in JP-A-2004-81745 may be adopted in the method for calculating the number of steps from the measured acceleration change.

The gyro sensor measures an angular velocity per unit time by using each X-axis, Y-axis, and Z-axis as a central axis in the acceleration sensor. The body motion information detecting unit 120 calculates an exercise intensity, and an exercise amount depending on a change amount as body motion information, by using the detected acceleration change data and the detected angular velocity data with the acceleration sensor and the gyro sensor, and integrating the change amounts thereof.

The body motion information detecting unit 120 may further include a global positioning system (GPS) sensor that detects position information.

The GPS sensor includes a GPS receiving function, and a position information calculating circuit, and measures the position information (latitude, longitude, and altitude) data per unit time. The body motion information detecting unit 120 may calculate a moving distance, and a moving velocity from the position information per unit time, and may calculate the exercise amount as body motion information by combining the values with the information of other sensors.

Moreover, the body motion information detecting unit 120 may include a sensor that detects a body temperature or blood pressure.

The communication unit 130 performs the wireless communication with a communication unit 205 of the portable terminal apparatus 200, and sends the pulse information detected by the pulse information detecting unit 110, and the body motion information detected by the body motion information detecting unit 120 to the portable terminal apparatus 200.

A wireless communication method is assumed to be Bluetooth (registered trademark), but is not limited to thereto. For example, various short-range wireless communication methods such as ZigBee (registered trademark), Wi-SUN (registered trademark), and IP500 (registered trademark) may be adopted.

Next, the functional units of the portable terminal apparatus 200 will be described.

The portable terminal apparatus 200 includes the communication unit 205, an operation accepting unit 210, a storage unit 215, a control unit 250, and a display unit 220.

The communication unit 205 is equivalent to a receiving unit, and receives the pulse information and the body motion information which are sent from the communication unit 130 of the wearable apparatus 100, and sends the received pulse information, and the received body motion information to the control unit 250.

The operation accepting unit 210 accepts an operation by a user, and sends a signal depending on the accepted operation to the control unit 250.

The display unit 220 displays an image which is generated by the control unit 250. In the embodiment, the portable terminal apparatus 200 includes a touch panel (not illustrated), and the touch panel is equivalent to the operation accepting unit 210 and the display unit 220.

The storage unit 215 is assumed to be a flash memory or the like as hardware, and stores the data or a program which is processed by the control unit 250.

The control unit 250 includes a pulse rate information obtaining section 255, an exercise information obtaining section 260, an additional information obtaining section 265, a calculation section 270, and a display control section 275.

Moreover, the control unit 250 performs processing or control of various sorts of information, and is assumed to be a processor as hardware. The respective functional units included in the control unit 250 are realized by cooperation of the storage unit 215 that stores the information of the program or the like, and the processor that operates based on the information of the program or the like stored in the storage unit 215.

The pulse rate information obtaining section 255 obtains pulse rate information from the pulse information which is sent from the wearable apparatus 100, and sends the obtained pulse rate information to the calculation section 270.

The exercise information obtaining section 260 obtains exercise information from the body motion information which is sent from the wearable apparatus 100, and sends the obtained exercise information to the calculation section 270.

The calculation section 270 calculates (determines) a minimum value (referred to as minimum pulse rate) of the pulse rate which becomes a standard for determining a health state or the like, namely, the pulse rate at the time of sleeping, based on the pulse rate information sent from the pulse rate information obtaining section 255, and the exercise information sent from the exercise information obtaining section 260. For example, a method disclosed in JP-A-2015-131049 may be adopted in the method for calculating the minimum pulse rate.

In the embodiment, the minimum pulse rate is a calculation target as analysis information, but it is not limited thereto. A resting pulse rate, a maximum pulse rate, a stress degree, a fatigue degree or the like may be the calculation target.

Furthermore, the calculation section 270 manages the pulse rate information sent from e pulse rate information obtaining section 255 in a time series, and calculates a basal pulse rate indicating the minimum value of the pulse rate over a predetermined long period such as several months. Moreover, it is known that the basal pulse rate appears when a body condition is fine or in a case where the user can soundly sleep.

The information of the minimum pulse rate or the basal pulse rate which is calculated by the calculation section 270 is stored in the storage unit 215 by being linked to the information of the calculated date and time.

The additional information obtaining section 265 obtains additional information relating to the minimum pulse rate, based on the operation which is accepted by the operation accepting unit 210. In the embodiment, the additional information is assumed to be an icon which is linked to the minimum pulse rate, or a comment relating to the minimum pulse rate. The additional information obtaining section 265 obtains the additional information which is generated by the operation of the touch panel with the user, and sends the obtained additional information to the display control section 275.

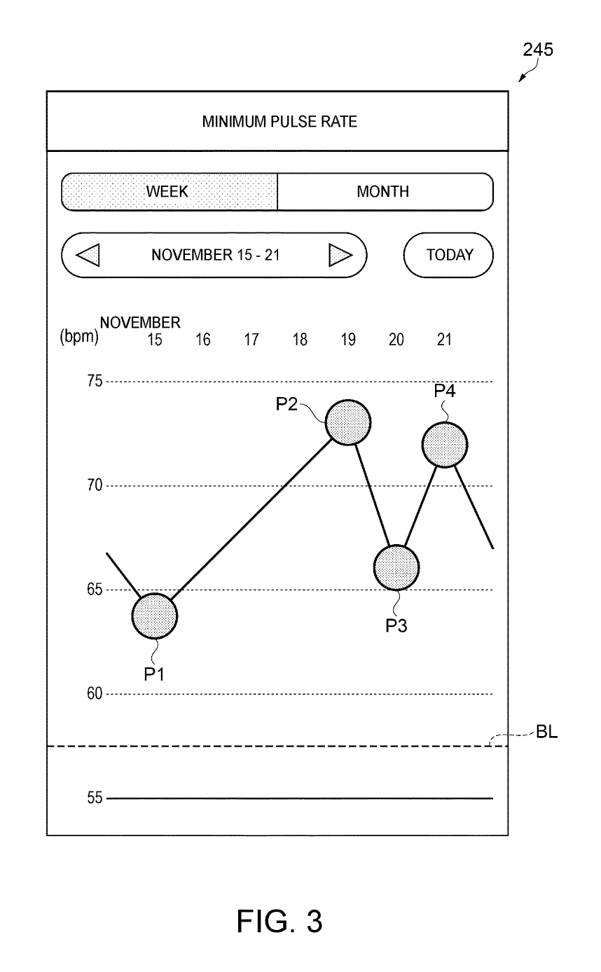

The display control section 275 controls a display of the image in the display unit 220. For example, FIG. 3 is an example of a transition image 245 of the minimum pulse rate which is displayed in the display unit 220. The display control section 275 generates the transition image 245 based on a predetermined operation of the user, and displays the generated transition image 245 in the display unit 220.

The display control section 275 assigns the value of the minimum pulse rate to one axis (vertical axis) of a graph, and time information such as date and time at which the minimum pulse rate is detected to the other axis (horizontal axis) of the graph, and generates the transition image 245 indicating transition of the minimum pulse rate by lapse of time. In the embodiment, the display control section 275 reads a plurality of the minimum pulse rate values which are stored in the storage unit 215, and the date and time information responding thereto, and superimposes the values on the transition image 245, and plots the value of the minimum pulse rate as a circle-shaped plot (P1 to P4) in the time series depending on the date and time information, and complements between the plots (P1 to P4) by a straight line. Furthermore, the display control section 275 reads the basal pulse rate stored in the storage unit 215, and causes a base line EL to be displayed by being superimposed on the transition image 245.

Moreover, the display control section 275 rewrites the transition image 245 depending on the operation, in a case where the user performs the operation of the operation accepting unit 210 by a finger or the like, with respect to the transition image 245 which the display unit 220 displays.

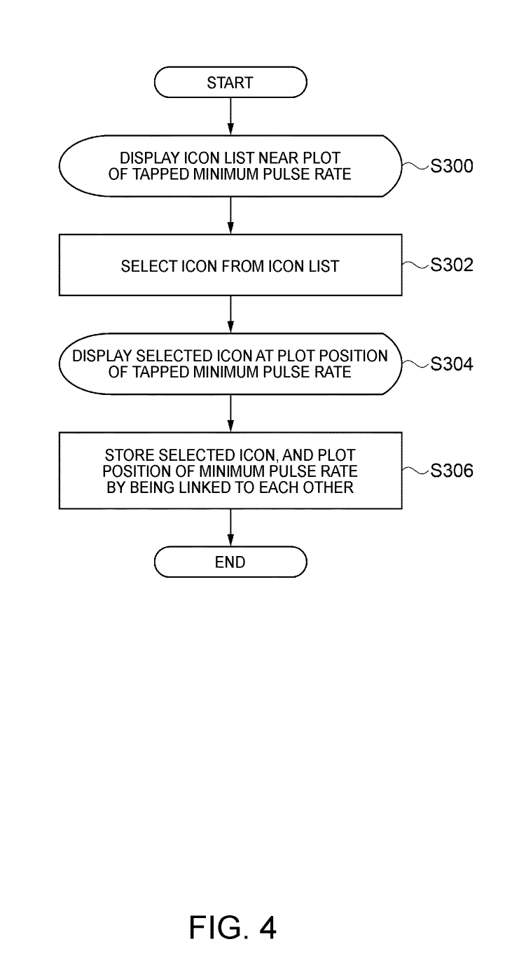

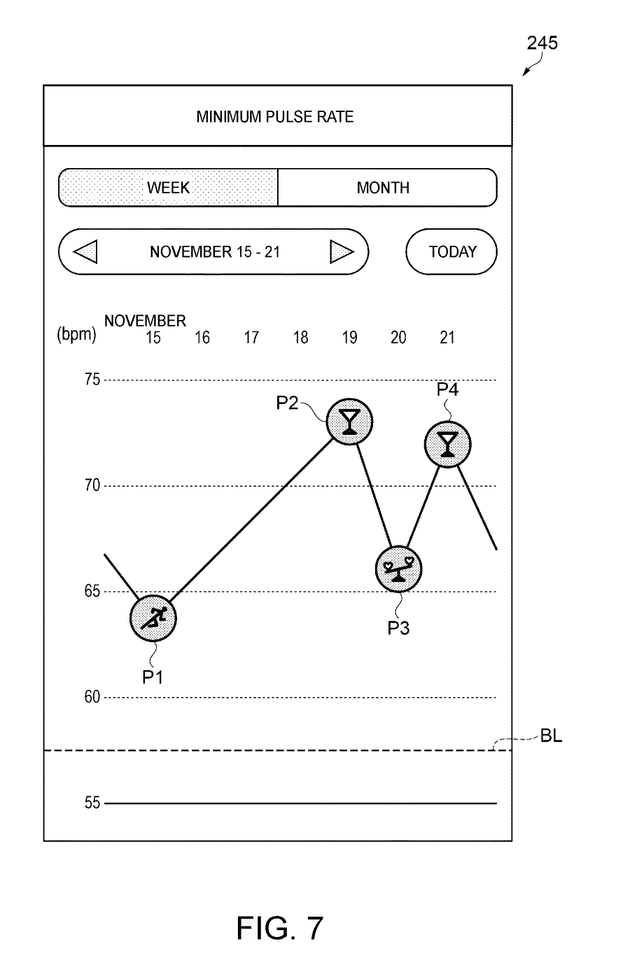

FIG. 4 is an example of a case where the transition image 245 is rewritten depending on the operation, and is a flowchart illustrating a flow of the processing that is executed in a case where the plot of the minimum pulse rate is tapped in the transition image 245, and a series of processing will be described. Moreover, the transition image 245 of each processing will be appropriately described, with reference to the respective transition images 245 in 5 to FIG. 7. The processing is equivalent to a biological information display method and a biological information display program.

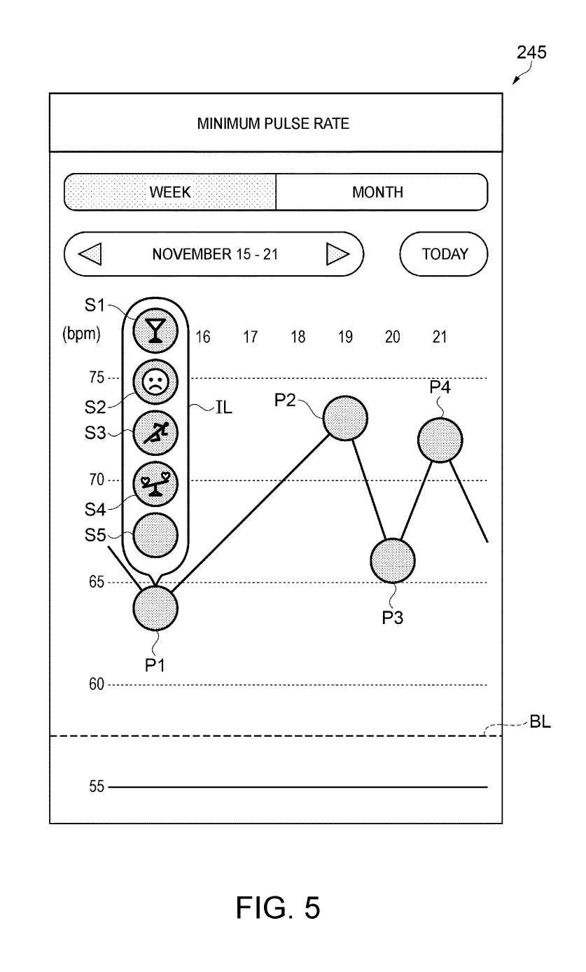

If the processing is started, the control unit 250 causes an icon list to be displayed near the plot of the tapped minimum pulse rate (step S300). As a result, an icon list IL is displayed near a plot P1 of the minimum pulse rate of the transition image 245 by being superimposed, as illustrate in FIG. 5.

The icon list IL includes at least one or more of the icons (S1 to S5) indicating subjective information indicating a subjective state of the user or event information relating to the user, as input information which is selectable information by the user. In the embodiment, an icon S1 is an icon indicating “to do a drink”. An icon S2 is an icon indicating “bad body condition”. An icon S3 is an icon indicating “to do exercise”. An icon S4 is an icon indicating “stress”. Moreover, an icon S5 is an icon to which meaning is not given, and it may be set to give the meaning and an icon design by the operation of the operation accepting unit 210 with the user. Furthermore, icons indicating a mental state, sleep time, and sleep depth may be included.

Next, a desired icon is selected from the icon list IL by the operation of the use (step S302). If the desired icon is selected from the icon list IL, the icon list IL is non-displayed. In the embodiment, a case where the icon S3 is selected as a desired icon is assumed.

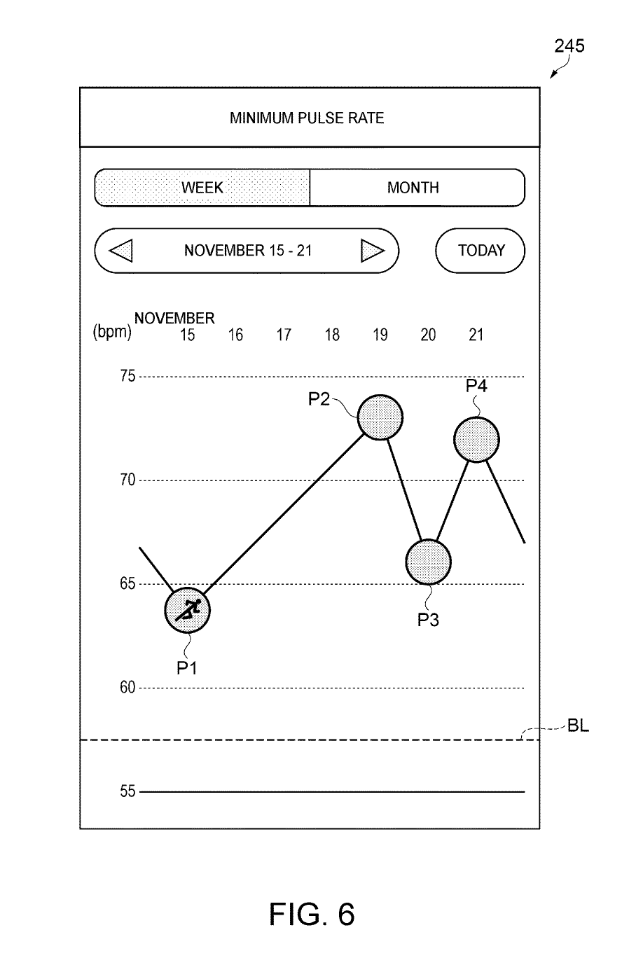

Next, the control unit 250 causes the selected icon S3 to be displayed by being superimposed, at a position of the plot P1 of the tapped minimum pulse rate (step S304). As a result, the selected icon S3 is displayed at the position of the plot P1 of the minimum pulse rate in the transition image 245, as illustrated in FIG. 6.

Next, the control unit 250 stores the selected icon S3, and the position of the plot P1 of the tapped minimum pulse rate in the storage unit 215 by linking the icon S3 to the position of the plot P1 (step S306). A series of processing is ended. Thereby, the icon which is linked to the plot of the transition image 245 is stored, and in a case where the transition image 245 is displayed again in the display unit 220, the icon which is linked to the position of each plot also displayed.

By tapping each plot (P2 to P4) of the minimum pulse rate, and respectively selecting the icons, the transition image 245 as illustrated in FIG. 7 is obtained. The user can visually recognize a case where the minimum pulse rate is close to the basal pulse rate by performing the exercise, a case where the minimum pulse rate is increased at the time of drinking, or a case where the minimum pulse rate is increased due to stress, from the transition image 245. Therefore, the user can have motivation to make a habit of exercising, motivation to set a nondrinking day, namely, a liver rest day, or motivation to take enough sleep or rest.

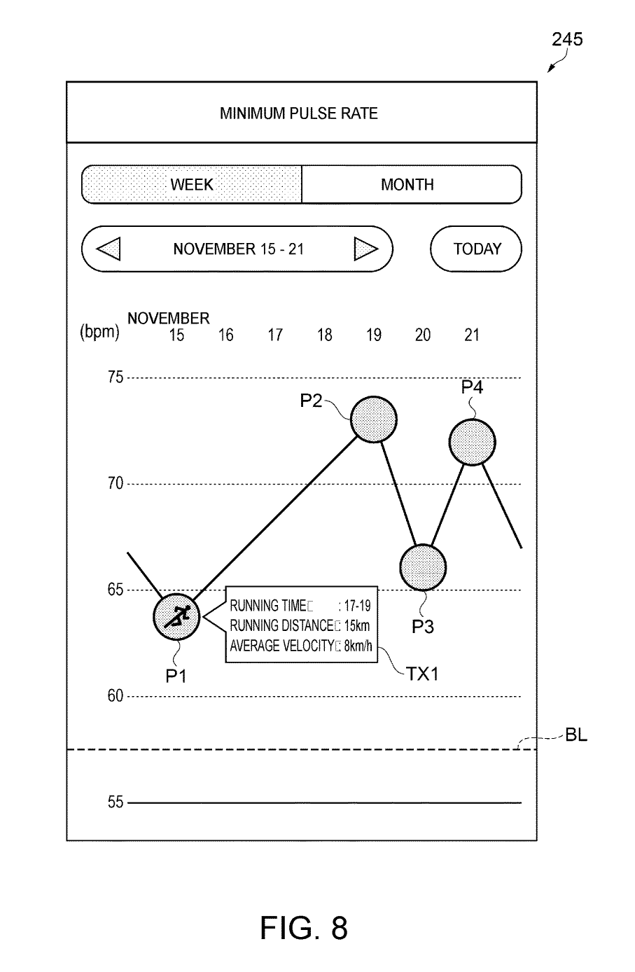

As illustrated in FIG. 8, for example, in a case where the plot P1 in which the icon 33 is displayed is pressed down, the control unit 250 displays a text input screen TX1 in the transition image 245. The user can input the detailed information or the comment of the icon S3. The control unit 250 stores the input content in the storage unit 215, and displays the content by reading the content if necessary.

In the transition image 245, a date or a period (week or month) at which the transition of the minimum pulse rate is displayed may be modified by the operation of the operation accepting unit 210 with the user.

According to the embodiment described above, the following effects are achieved.

The control unit 250 of the portable terminal apparatus 200 generates the transition image 245 indicating the transition of the minimum pulse rate of the user, and causes the generated transition image 245 to be displayed in the display unit 220. If the user taps the plot P1 responding to the minimum pulse rate in the transition image 245, the control unit 250 causes the icon list IL including at least one of the subjective information relating to user and the event information relating the user to be displayed near the plot P1 of the minimum pulse rate in the transition image 245. In response to this, in a case where the user selects the desired icon S1 from the icon list IL, the selected icon S1 is displayed by being superimposed at the position of the tapped plot P1. Accordingly, the user can estimate a factor for the tapped minimum pulse rate based on the displayed icon S1. As a result, the user can estimate the factor which influences the body condition or the like of the self, and can take a countermeasure in order to improve the body condition.

Hitherto, the suitable embodiments are described with reference to the accompanying drawings, but the suitable embodiments are not limited to the embodiments described above. The embodiments can be variously modified within the scope without departing from the gist, needless say, and can be carried out as follows.

For example, a state where a server apparatus (of which the illustration is omitted) is included in the biological information display system 50, and the server apparatus connected to the wearable apparatus 100 or the port able terminal apparatus 200 through the communication may be also assumed.

In general, the server apparatus has high processing performance in comparison with the wearable apparatus 100 or the portable terminal apparatus 200, Therefore, the server apparatus has some of the functions which the control unit 250 of the portable terminal apparatus 200 has, for example, the function of the calculation section 270, and thereby, it is possible to perform various sorts of processing at a high velocity, in comparison with a case where the portable terminal. apparatus 200 has the functions. Since the server apparatus has large storage capacity, it is possible to store the pulse rate or the exercise information by unit of several years several decades. Thereby, the calculation or the analysis is possible based on the data of the long period, and it is possible to expect improvement in reliability of the basal pulse rate, or improvement in analysis accuracy relating to the transition of the minimum pulse rate.

Moreover, a state where the wearable apparatus 100 has the function of the portable terminal apparatus 200, and the biological information display system 50 is realized by only the wearable apparatus 100 may be assumed. That is, the wearable apparatus 100 includes the operation accepting unit 210, the storage unit 215, the control unit 250, and the display unit 220, in addition to the configuration described above. Thereby, it is possible to make miniaturization and weight reduction of the biological information display system 50.

The selectable icon which is displayed in the icon list IL is not limited thereto. For example, the user presses down the icon S5, and thereby, the subjective body condition may be defined in the icon S5.

The base line EL of the basal pulse s displayed by being superimposed in the transition image 245, but is not limited thereto. The control unit 250 may generate the base line of the minimum pulse rate based on the body condition or the sleep state in a case where the body condition or the sleep state of the user is fine, and may cause the generated base line to be displayed by being superimposed in the transition image 245. In this case, the base line may be set by the user, or may be generated by the control unit 250 based on a body condition situation or a sleep situation.

The control unit 250 may calculate a divergence degree between the base line of the generated minimum pulse rate and the current minimum pulse rate, and may determine the fatigue degree of the user based on the calculated result. Furthermore, the control unit 250 may include a notification unit (of which the illustration is omitted) that makes a message or advice on the user based on the input content which is input to the text input screen TX1 or the divergence degree, and notifies the user of the message or the advice.

The control unit 250 may add an added value by combining the minimum pulse rate with other data. For example, it is possible to realize a correlation between the resting situations of the body in a case of sleeping at home and a case of sleeping in a hotel, by combining the transition of the minimum pulse rate of the user with the position information of the user. As other data, stress (HRV), the number of steps, exercise time, standing time, and sitting time may be assumed.

The information which is displayed by pressing down the plot P1 is not limited to the text input screen TX1. For example, if the plot P1 is pressed down, a program responding to the displayed icon may be booted, and the program may obtain the information linked to the icon, and may be displayed in the display unit 220. For example, the program may access an external server or the like, and may display various sorts of the obtained information.

For example, in a case of the icon S1 indicating “to do a drink”, dietary composition which the user ingests, a dietary intake, a meal time or the like may be obtained.

In a case of the icon S2 indicating “bad body condition”, the body motion information detecting unit 120 may obtain the body temperature information or the blood pressure information of the user. For example, an air temperature, weather, pollen dispersal information, influenza epidemic information or the like may be obtained from an external server apparatus.

In a case of the icon S3 indicating “to do exercise”, the body motion information detecting unit 120 may obtain load information such as pulse zone staying time, exercise classification, and exercise time.

Moreover, in a case of the icons S4 indicating “stress”, time, intensity, place, schedule or event in a case where stress is increased, and time, intensity, place, schedule, or event in a case of being relaxed, and the number of plots of an event marker on a map may be obtained.

The program may display a separate window in the display unit 220, and the obtained information may be selected by the user.

The respective functional units of the control unit 250 illustrated in FIG. 2, are functional units illustrating the functional configuration that is realized by cooperation of hardware and software, and a specific installation form thereof not particularly limited. Accordingly, the hardware which separately responds to each functional unit is not necessarily installed, and a configuration in which functions of a plurality of functional units are realized by executing a program with one processor is possible, needless to say.

In the embodiments described above, some of the functions which are realized by the software may be realized by the hardware, or some of the functions which are realized by the hardware may be realized by the software. In addition, the specific detailed configuration of each of other units of the control unit 250 may be optionally modified within the scope without departing from the gist of the invention.

The transition indicating a time series change of the minimum pulse rate, and the transition of each data (for example, the data indicating the subjective body condition or the data of the biological information) have the correlation with a life pattern of the user. Accordingly, in a case where a pattern (second change pattern) including the transition of the most recent minimum pulse rate and the transition of each data, is matched with a pattern (first change pattern) indicating the past data stored in the server apparatus or the storage unit 215, the control unit 250 may notify the user of a state where the matched past data is found as a push notification.

Furthermore, the server apparatus may evaluate the matched similarity degree, and may predict the future transition, and may strongly prompt the user to modify a behavior by displaying the advice or the message in the wearable apparatus 100. Thereby, the user can prevent a deterioration of the body condition in advance.

What is claimed is:

1. A biological information display system comprising:
a display unit;
a biological information obtaining unit that obtains biological information of a user;
a control unit that determines analysis information relating to a state of the user based on the biological information, and causes the analysis information, and time information responding to the analysis information to be displayed in the display unit by being linked to each other; and
an accepting unit that accepts input information including at least one of subjective information relating to the user and event information relating the user by associating the input information with the time information,
wherein in a case where the accepting unit accepts the input information, the control unit causes the input information to be displayed in the display unit by being linked to the analysis information and the time information.

2. The biological information display system according to claim 1,
wherein the control unit causes a plurality of pieces of the analysis information to be displayed in a time series depending on respective pieces of the time information.

3. The biological information display system according to claim 1,
wherein the input information is information relating to at least one of drinking, a body condition, exercise, a mental state, sleep time and sleep depth.

4. The biological information display system according to claim 1,
wherein the biological information is at least one of pulse wave information, electrocardiac information and a pulse rate.

5. The biological information display system according to claim 1,
wherein the analysis information is at least any one of a minimum pulse rate, a resting pulse rate, a maximum pulse rate, a stress degree and a fatigue degree.

6. The biological information display system according to claim 1,
wherein the control unit assigns the analysis information to one axis of a graph and the time information to the other axis of the graph, generates a transition image indicating transition of the analysis information by lapse of time, and causes the transition image to be displayed in the display unit.

7. The biological information display system according to claim 6,
wherein the control unit indicates the analysis information depending on the time information by a plot in the transition image, and causes an icon depending on the input information to be displayed by being superimposed on the plot in a case where the input information is linked to the time information.

8. The biological information display system according to claim 6,
wherein the analysis information is a minimum pulse rate, and
the control unit determines a basal pulse rate of the user based on transition of the minimum pulse rate, and indicates a base line indicating the basal pulse rate on the transition image.

9. The biological information display system according to claim 6,
wherein the analysis information is a minimum pulse rate, and
the control unit obtains the minimum pulse rate in a case where a body condition or a sleep state of the user is fine, and indicates a base line indicating the minimum pulse rate on the transition image.

10. The biological information display system according to claim 8,
wherein the control unit determines a fatigue degree of the user based on a divergence degree between the minimum pulse rate and the base line.

11. biological information display system according to claim 10,
wherein the control unit includes a notification unit that notifies the user of a message, based on the determined fatigue degree and the input information.

12. The biological information display system according to claim 1,
wherein the control unit evaluates a similarity degree between a first change pattern indicating a time series change of the analysis information and a second change pattern of the analysis information including most recent information, and predicts a change relating to a fatigue degree of the user based on the similarity degree.

13. A portable terminal apparatus comprising:
a display unit;
a receiving unit that receives biological information of a user;
a control unit that calculates a pulse rate of the user based on the biological information, and determines analysis information based on the calculated pulse rate, and causes the analysis information, and time information responding to the analysis information to be displayed in the display unit by being linked to each other; and
an accepting unit that accepts input information including at least one of subjective information relating to the user and event information relating to the user by associating the input information with the time information,
wherein in a case where the accepting unit accepts the input information, the control unit causes the input information to be displayed in the display unit by being linked to the analysis information and the time information.

14. A wearable apparatus comprising:
a display unit;
a biological information obtaining unit that obtains biological information of a user;
a control unit that determines analysis information relating to a state of the user based on the biological information, and causes the analysis information, and time information responding to the analysis information to be displayed in the display unit by being linked to each other; and
an accepting unit that accepts input information including at least one of subjective information relating to the user and event information relating to the user by associating the input information with the time information,
wherein in a case where the accepting unit accepts the input information, the control unit caul the input information to be displayed in the display unit by being linked to the analysis information and the time information.

15. A biological information display method comprising:
obtaining biological information of a user which is measured by a sensor;
determining analysis information relating to a state of the user based on the biological information, and displaying the analysis information, and time information responding to the analysis information in a display unit by linking the analysis information to the time information; and
displaying input information by linking the input information to the time information, if obtaining the input information including at least one of subjective information relating to the user and event information relating to the user which respond to the analysis information from an accepting unit.

16. The biological information display method according to claim 15,
wherein the input information is displayed by being linked to the analysis information and the time information, if the input information is obtained from the accepting unit.

17. The biological information display method according to claim 15,
wherein a plurality of pieces of the analysis information is displayed in a time series.

18. The biological information display method according to claim 15,
wherein the input information is information representing at least one of drinking, a body condition, exercise, a mental state, sleep time and sleep depth.

19. The biological information display method according to claim 15,
wherein a graph where one axis is the analysis information, and the other axis is the time information, and transition of the analysis information is indicated by lapse of time is displayed.

20. The biological information display method according to claim 19,
wherein the input information includes an icon which is selected by the user, and
the icon is displayed at a position responding to the time information or the analysis information in the graph.
